(19) United States
(12) Patent Application Publication
(10) Pub. No.: US 2019/0221293 A1
(43) Pub. Date: Jul. 18, 2019

(54) ANALYSIS AND MANAGEMENT METHOD FOR PHARMACEUTICAL EXCIPIENTS

(71) Applicant: Tai ZHANG, SHANDONG (CN)

(72) Inventor: Tai ZHANG, SHANDONG (CN)

(21) Appl. No.: 16/307,005

(22) PCT Filed: Sep. 14, 2017
(86) PCT No.: PCT/CN2017/090981

§ 371 Date: Dec. 4, 2018

(30) Foreign Application Priority Data
Sep. 29, 2016 (CN) 201610866272.9

(51) Int. Cl. G16C 20/70

(52) U.S. Cl. CPC G16C 20/70

(57) ABSTRACT

A method for analyzing and managing a pharmaceutical excipient is provided. By collecting data of pharmaceutical excipients and pharmaceutical products to establish a database, associating related websites, and updating the data in real-time, the method integrates research resources of the pharmaceutical excipients to provide a safe pharmaceutical administration service, and enables acquisition of a rapid and accurate pharmaceutical administration analysis result after recipient information is inputted. Various types of vehicles and terminals can be applied for operations. The embodiment can be utilized independently on computers, communications equipment, or mobile data hand-held terminal devices, or can be integrated into a pharmaceutical administration management system or embedded into a server of a medical facility to be used alongside a medical service system, thus eliminating a large amount of tedious manual monitoring operations, improving working efficiency, reducing labor intensity, and providing superior pharmaceutical administration safety management.

### CROSS-REFERENCE TO RELATED APPLICATIONS

This application claims priority to PCT Application No. PCT/CN2017/090981, having a filing date of Sep. 14, 2017, based on Chinese Application No. 201610866272.9, having a filing date of Sep. 29, 2016, the entire contents both of which are hereby incorporated by reference.

### FIELD OF TECHNOLOGY

The following belongs to the technical field of drug application analysis, and more specifically, relates to an analysis and management method for pharmaceutical excipients where the medication safety is analyzed based on the characteristics of pharmaceutical excipients, and especially, an early warning management is carried out for potential adverse consequences before the use of drugs so as to guarantee the safe and appropriate use of drugs.

### BACKGROUND

Drugs are substances used to prevent, treat and diagnose human diseases and purposefully regulate human physiological functions and prescribed with indications or major functions, usage and dosage, including Chinese medicinal materials, Chinese herbal slices, Chinese traditional patent medicine, chemical active pharmaceutical ingredients (medicinal/pharmaceutical chemicals) and preparations thereof, antibiotics, biochemical drugs, radioactive drugs (radiopharmaceuticals), serum, vaccines, blood products and diagnostic drugs. There are complex types and many varieties of drugs, and over 20000 varieties around the world. The “SFDA Approval Number H (or Z, S, J, B, F)+8 digits” printed on drug packages indicates that this drug is produced, marketed and sold upon approval by the China Food and Drug Administration, wherein H represents chemical drugs, Z represents Chinese traditional patent medicine, S represents biological products, J represents repackaging in China of imported drugs, B represents drugs having adjuvant therapeutic effects, and F represents pharmaceutical excipients. Pharmaceutical excipients are all medicinal materials except sovereign drugs that are added into prescriptions to solve the formability, efficacy, stability and safety of preparations in design of preparation prescriptions, including vehicles and additives used in producing drugs and dispensing prescriptions. In addition to shaping, acting as carriers and enhancing stability, pharmaceutical excipients also have important functions of solubilization, hydrotrop, sustained and controlled release, and they are important ingredients which may affect the quality, safety and efficacy of drugs. Pharmaceutical excipients are not only the basic materials and important integral parts of pharmaceutical preparations, but also the material base for production and development of pharmaceutical preparations, since they play a key role in dosage forms and the production of preparations where pharmaceutical excipients not only give drugs certain forms, but also make a great difference in enhancing drug efficacy and reducing adverse effects, and their quality reliability and diversity are the basis for the advancement of dosage forms and preparations. Pharmaceutical excipients are classified into 66 types by their function in preparations, including solvents, propellants, solubilizers, hydrotropy agents, emulsifiers, colorants, binders, disintegrants, fillers, lubricants, wetting agents, osmotic pressure regulating agents, stabilizers, glidants, flavoring agents, preservatives, suspending agents, coating materials, aromatics, antiadherents, integrating agents, penetration enhancers, pH regulators, buffers, plasticizers, surfactants, blowing agents, defoamers, thickeners, inclusion agents, humectants, absorbents, diluents, flocculating agents, deflocculating agents, filter aids, release blocking agents, etc. All analysis and management of drug use in the prior art is for the sovereign drugs in drugs, i.e. predictions in safe use of drugs are made only for the pharmacological and toxicological properties of active ingredients. When drugs are used in combination, the pharmaceutical excipients and the sovereign drugs in the drugs jointly act on the human body and participate in the whole pharmacokinetic process of the drugs, and these pharmaceutical excipients produce important effects on the results of drug use based on their own characteristics. Pharmaceutical excipients not only have certain pharmacological and toxicological activities, but also affect the physical and chemical properties of drugs, the compatibility when multiple drugs are used together, and affect the pharmacokinetic parameters and clinical examination results of the drugs. The current common combined use and sequential use of multiple drugs increases the opportunities of interactions between different pharmaceutical excipients as well as the pharmaceutical excipients and sovereign drugs of different drugs, and also increase the possibility of drug incompatibility causing adverse consequences; there are no patent or literature on the analysis and management of the effects of the characteristics of pharmaceutical excipients on the safety of drug use. Therefore, an analysis and management method for pharmaceutical excipients is developed where the safety of the mixed use of drugs is analyzed based on the characteristics of pharmaceutical excipients, and especially, an early warning management is carried out for potential adverse consequences before the use of drugs, to guarantee safe and appropriate use of drugs, having a prospect for market promotion and application values.

### SUMMARY

An aspect relates to an analysis and management method for pharmaceutical excipients where the safety of drug use is analyzed based on the characteristics of the pharmaceutical excipients, and especially before the use of drugs, an accurate and quick early warning is made of potential adverse consequences which the pharmaceutical excipients in the drugs have for prevention, diagnosis, treatment and rehabilitation activities by using computers or other mobile devices as the carrier based on the study of the pharmaceutical excipients to guarantee the safe and appropriate use of drugs.

The analysis and management method for pharmaceutical excipients according to embodiments of the present invention is realized on-line or stand-alone in the server, computer, communication equipment or handheld mobile data terminals of the medical organization equipped with an analysis and management apparatus for pharmaceutical excipients, whose process includes four steps: creation of a database, input of the information on the drug user, Analysis of the information on the drug user and feedback of the analysis results:

(1) Creation of a database: the data on the drugs and pharmaceutical excipients currently used in the market are input into an information input unit by scanning QR codes, entry or online download, the information input unit sends the data above to the information processing unit for recording and storage, the data above is regularly maintained, updated, modified, supplemented and deleted, and the data above is associated to realize the creation of a database;
The data input include review, pharmaceutical research materials, dietetic contraindications of pharmaceutical excipients, and incompatibilities between pharmaceutical excipients as well as between pharmaceutical excipients and sovereign drugs; the review includes the name and nomenclature basis of drugs or pharmaceutical excipients, supporting documents, purposes and basis of subject determination, summary and evaluation of main research results, a sample, preparation instructions and the latest references of inserts, and design samples of packages and labels; the pharmaceutical research materials include the review of the pharmaceutical research materials, the research materials and literature materials on production processes, the test materials and literature materials on confirmation of chemical structures or constituents, the test materials and literature materials on quality research, the compatibility test materials and literature materials on drugs, standard drafts and preparation instructions, standards or controls, the self-inspection reports of three consecutive lots of drugs, the test materials and literature materials on stability studies, the selection basis and quality standard constitution of packaging materials and containers in direct contact with pharmaceutical excipients, as well as periodical literature, newspaper reporting, research reports and Internet information covering adverse consequences caused by the properties of pharmaceutical excipients;
wherein the name of drugs or pharmaceutical excipients includes a formal product name, chemical name, English name and Pinyin; the supporting documents include documents supporting the legal registration of the applicant, the copy of the Drug Production License, the description of the patent situation and ownership state of the prescription and processes of pharmaceutical excipients or used as well as a guarantee against the infringement of others patents; the purposes and basis of subject determination include foreign and domestic literature materials and situation review relevant to research and development, entry into the market and sales, production and application in preparations of this product; the summary and evaluation of the main research results include the summary of the main research results and the comprehensive evaluation of the reported variety in safety, efficacy and quality controllability; a sample, preparation instructions and the latest references of inserts include names, chemical structural formulae or molecular formulae, uses, precautions, packages, specifications, contents and the expiry date of pharmaceutical excipients and a clear indication of pharmaceutical excipients; the review of the pharmaceutical research materials the overview of tests materials and foreign and domestic literature materials on synthesis processes, proscription screening, structure confirmation, quality research, quality standard development, stability studies, etc.; the research materials and literature materials on production processes include the process flow, chemical reaction formulae, operation steps, starting materials, organic solvents, reaction conditions (temperature, pressure, time and catalysts) and operation steps of preparation, refinement methods, main physical and chemical constants as well as data accumulation results at different stages, indication of feeding amount and yield rate, possible impurities or other intermediate products produced or introduced during the processes, and provision of the specifications and standards of chemical raw materials used, the source, scientific name, preparation process and modification basis (where any main references are different) of animal-based, plant-based and mineral-based raw materials; the test materials and literature materials on quality research include physical and chemical constants, purity testing, content determination and methodological validation as well as data accumulation results at different stages; the test materials and literature materials on drug compatibility, standard drafts and preparation instructions, and standards or controls;
(2) Input of the information on the drug user: the information on the drug user and the prescription or the drug name of the doctor's advice are input on-line or stand-alone into the information input unit which supports QR code scanning, links and entry and has a drop-down menu; Wherein the information on the drug user includes the physical examination report, past medical history, history of present illness, examination and treatment situations, description and parameters about conditions, and information about sovereign drugs and minister drugs of the used drugs of the drug user;
(3) Analysis of the information on the drug user: after receiving the information on the drug user, the information processing unit searches the information on the sovereign drugs and minister drugs in the prescription or the drug of the doctor's advice, makes a comparison with the information in the database, analyzes whether there exist potential adverse consequences, and if there are any adverse consequence, searches in the database for drug substitutes equivalent to the sovereign drugs in the prescription or the drug of the doctor's advice without changing the drug use intention, and makes an analysis to obtain an optimized safe plan of the prescription or the drug of the doctor's advice in which the sovereign drugs of the drug substitutes attain the drug-use intention and there doesn't exist any adverse consequence between the minister drugs of the drug substitutes and other prescriptions or drugs of the doctor's advice;
(4) Feedback of analysis results: the information processing unit sends the optimized safe plan of the prescription or the drug of the doctor's advice obtained by analysis to the window display unit for the operator's use as reference through a result feedback unit.

The main structure of the analysis and management apparatus for pharmaceutical excipients according to embodiments of the present invention comprises an information input unit, an information processing unit, a result feedback unit and a window display unit, wherein wireless electrical connection is adopted between the information input unit and the information processing unit, the information processing unit and the result feedback unit as well as the result feedback unit and the window display unit, the information input unit supports manual input, on-line input and input by scanning QR codes, the information processing unit records, stores, analyzes and manages the information input by the information input unit, and the result feedback unit sends the information analyzed and processed by the information processing unit to the window display unit, and the window display unit shows the results fed back by the result feedback unit.

The principle of the analysis and management apparatus for pharmaceutical excipients according to embodiments of the present invention: relevant medical research shows that pharmaceutical excipients that were previously regarded as inactive actually have obvious pharmacological actions; some have been incorporated into the chemical drug directory of the Chinese Pharmacopoeia; pharmaceutical excipients can be used as drugs and are not inert components, and their pharmacological actions and physical and chemical parameter characteristics can change pharmacokinetic parameters, cause other diseases, reduce the reliability of diagnosis methods or testing results, cause or intensify toxic reactions, induce allergic reactions, make people more susceptible to diseases, worsen conditions and cause condition transfer and development after prognosis, so re-examination, analysis, management and early warning must be carried out for pharmaceutical excipients in order to ensure the safety of drug use.

The analysis and management apparatus for pharmaceutical excipients according to embodiments of the present invention provides analysis, management and early warning services for drug use behaviors from the perspective of the effects of the pharmaceutical excipients in drugs on prevention, diagnosis, treatment and rehabilitation activities, provides a new guarantee for safe and appropriate drug use, effectively avoid adverse consequences induced by the administration of drugs, and provides a safe optimized plan containing the same sovereign drugs as and different pharmaceutical excipients from the prescription or drug of the doctor's advice. This can attain the initial drug use intention, provide reference and help for the medical personnel of drug analysis and management, provide advice for the medical personnel of nursing, clinical testing and imaging, provide professional analysis feedback for the staff members of prevention, rehabilitation and social health and physical examination organizations, helpful for raising the professional level and safety of medical services, enabling ordinary users without the basic knowledge about drugs to analyze and manage specific drugs and bringing convenience for drug users with limited mobility and far away from medical organizations.

Compared with the prior art, in embodiments of the present invention, a database is established by collecting the data of pharmaceutical excipients and drugs, relevant websites are associated, data is updated in real time, research resources of drugs and pharmaceutical excipients are integrated to provide safe drug use service, and an accurate drug use analysis result can be quickly obtained after the information on the drug user is input, so embodiments of the present invention has reliability and development sustainability, enables ordinary users without the basic knowledge about drugs to analyze and manage specific drugs and brings convenience for drug users with limited mobility and far away from medical organizations. Various carriers and terminal forms can be used for operating the application, suiting the development trend of the society. The apparatus can be used alone on the computer, communication equipment or handheld mobile data terminals, and can also be integrated into the drug use management system or embedded in the server of a medical organization and used together with the medical service system, omitting a large amount of cumbersome monitoring work, improving work efficiency, reducing work intensity and realizing the drug-use safety management to the greatest extent; embodiments of the present invention features a scientific principle, a simple procedure, practical functions, simple operation, good safety performance, a flexible application mode and a friendly application environment.

### BRIEF DESCRIPTION

Some of the embodiments will be described in detail, with references to the following Figures, wherein like designations denote like members, wherein:

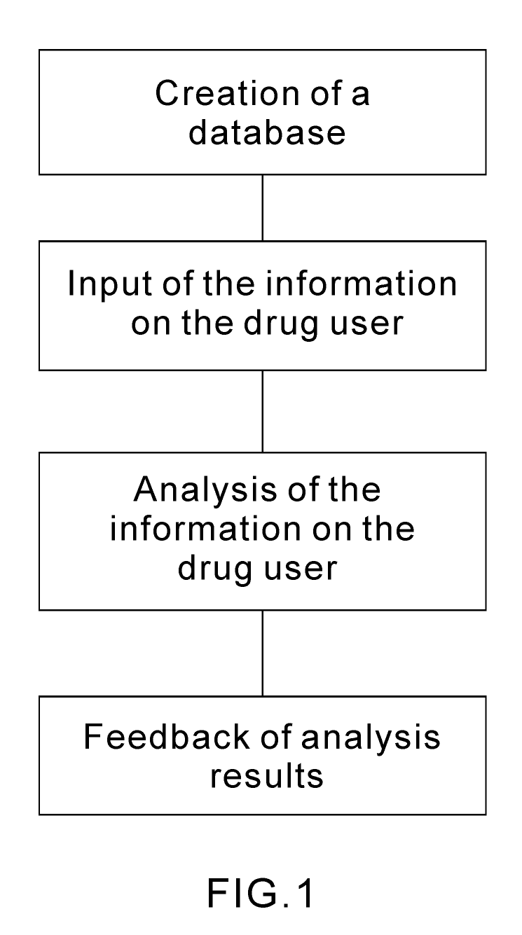

FIG. 1 is a block flow diagram of the principle of embodiments of the present invention; and

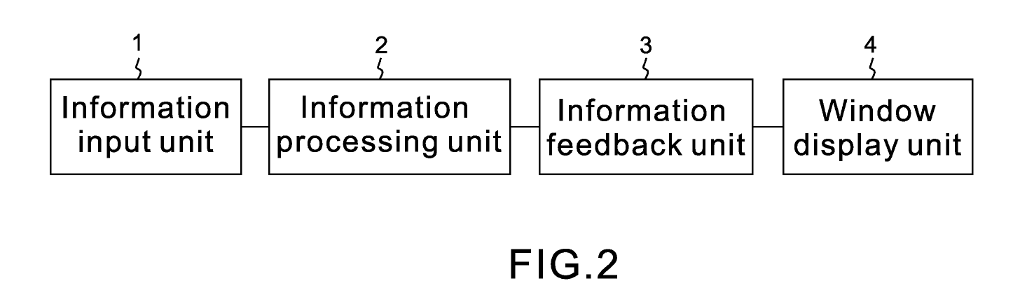

FIG. 2 is a principle and structural diagram of the analysis and management apparatus for pharmaceutical excipients.

### DETAILED DESCRIPTION

The following will now be described with some preferred embodiments thereof and by referring to the accompanying drawings.

### Embodiment 1

The embodiment relates to an analysis and management method for pharmaceutical excipients which is realized on-line in the server of the medical organization equipped with an analysis and management apparatus for pharmaceutical excipients, whose process includes four steps: creation of a database, input of the information on the drug user, analysis of the information on the drug user and feedback of analysis results:

(1) Creation of a database: the development personnel of the analysis and management apparatus for pharmaceutical excipients input the data on the drugs and pharmaceutical excipients currently used in the market into an information input unit 1 by scanning QR codes, entry or online download, the information input unit 1 sends the data above to the information processing unit 2 for recording and storage, the data above are regularly maintained, updated, modified, supplemented and deleted, and the data above are associated to realize the creation of a database;
(2) Input of the information on the drug user: the doctor inputs the information on the drug user and the prescription of the doctor's advice online into the information input unit 1. The information input unit 1 supports input by scanning QR codes, links and entry and provides a drop-down menu. The information on the drug user: premature boy born 3.5 h early at 26 weeks' gestational age, birth weight 0.83 kg, extremely premature infant, severe neonatal asphyxia, neonatal apnea, neonatal hypoglycemia, neonatal hyperglycemia, neonatal hypothermia, dysfunction of blood coagulation and mixed acidosis. The prescription of the doctor's advice: intravenous injection of human immunoglobulin 16.g ST ivgtt to give immune support;
(3) Analysis of the information on the drug user: after receiving the information on the drug user, the information processing unit 2 makes an analysis and management by associating and comparing the information on the drug user and the database, and finds that there are fluctuations in the blood sugar of the infant patient, that since admission to the hospital, the blood sugar has been regularly monitored using the blood sugar monitoring system adopting the glucose dehydrogenase pyrroloquinoline quinine (GDH-PQQ) enzyme technology, and the human immunoglobulin contains maltose, so the information processing unit 2 derives an early warning from the analysis that the blood sugar monitored by the blood sugar monitoring system may show false hyperglycemia after intravenous injection of human immunoglobulin;
(4) Feedback of analysis results: the information processing unit 2 sends the analysis result to the window display unit 4 for the doctor's use as a reference through the result feedback unit 3. The doctor analyzes the result and changes the doctor's advice or uses a different blood sugar monitoring system of a different operating mechanism according to the early warning information in the window display unit 4, so that the doctor will not be misled by the blood sugar caused by the pharmaceutical excipient and issue an incorrect prescription.

The main structure of the analysis and management apparatus for pharmaceutical excipients according to embodiments of the present invention comprises an information input unit 1, an information processing unit 2, a result feedback unit 3 and a window display unit 4, wherein wireless electrical connection is adopted between the information input unit 1 and the information processing unit 2, the information processing unit 2 and the result feedback unit 3 as well as the result feedback unit 3 and the window display unit 4, the information input unit 1 supports manual input, on-line input and input by scanning QR codes, the information processing unit 2 records, stores, analyzes and manages the information input by the information input unit 1, the result feedback unit 3 sends the information analyzed and processed by the information processing unit 2 to the window display unit 4, and the window display unit 4 shows the results fed back by the result feedback unit 3.

### Embodiment 2

The embodiment relates to an analysis and management method for pharmaceutical excipients which is realized on-line in the server of the medical organization equipped with an analysis and management apparatus for pharmaceutical excipients, whose process includes four steps: creation of a database, input of the information on the drug user, analysis of the information on the drug user and feedback of analysis results:

(1) Creation of a database: the development personnel of the analysis and management apparatus for pharmaceutical excipients input the data on the drugs and pharmaceutical excipients currently used in the market into an information input unit 1 by scanning QR codes, entry or online download, the information input unit 1 sends the data above to the information processing unit 2 for recording and storage, the data above are regularly maintained, updated, modified, supplemented and deleted, and the data above are associated to realize the creation of a database;
(2) Input of the information on the drug user: the doctor inputs the information on the drug user and the prescription of the doctor's advice online into the information input unit 1. The information input unit 1 supports input by scanning QR codes, links and entry and provides a drop-down menu. The information on the drug user: 3 year-old boy patient, body weight 15 kg, in a fever for 2 d, pharyngalgia and violent dry cough for 3 d, body temperature 39.5° C., and clinically diagnosed as community acquired pneumonia.

The prescription of the doctor's advice: intravenous drip of 5% glucose injection+prednisolone injection, once a day; intravenous drip of 0.9% sodium chloride injection+cefmetazole sodium for injection, twice a day; and intravenous drip of 0.9% sodium chloride injection+erythromycin lactobionate for injection;

(3) Analysis of the information on the drug user: after receiving the information on the drug user, the information processing unit 2 makes an analysis and management by associating and compares the information on the drug user and the database, and finds that the prednisolone injection in the doctor's advice contains a pharmaceutical excipient ethyl alcohol, so the information processing unit 2 derives from the analysis that there is a possible disulfiram-like reaction when the ethyl alcohol is used in combination with the cefmetazole sodium with a 5-mercapto-1-methyltetrazole (MTT) group;
(4) Feedback of analysis results: the information processing unit 2 sends the analysis result to the window display unit 4 for the doctor's use as reference through the result feedback unit 3. The doctor analyzes the result and changes the doctor's advice in time according to the early warning in the window display unit 4, so as to avoid the disulfiram-like reaction.

### Embodiment 3

The embodiment relates to an analysis and management method for pharmaceutical excipients which is realized on-line in the server of the medical organization equipped with an analysis and management apparatus for pharmaceutical excipients, whose process includes four steps: creation of a database, input of the information on the drug user, analysis of the information on the drug user and feedback of analysis results:

(1) Creation of a database: the development personnel of the analysis and management apparatus for pharmaceutical excipients input the data on the drugs and pharmaceutical excipients currently used in the market into an information input unit 1 by scanning QR codes, entry or online download, the information input unit 1 sends the data above to the information processing unit 2 for recording and storage, the data above are regularly maintained, updated, modified, supplemented and deleted, and the data above are associated to realize the creation of a database;
(2) Input of the information on the drug user: the doctor inputs the information on the drug user and the prescription of the doctor's advice online into the information input unit 1. The information input unit 1 supports input by scanning QR codes, links and entry and provides a drop-down menu. The information on the drug user: 28 year-old female drug user, luteal insufficiency. The prescription of the doctor's advice: intramuscular injection of chorionic gonadotrophin;
(3) Analysis of the information on the drug user: after receiving the information on the drug user, the information processing unit 2 makes an analysis and management by associating and comparing the information on the drug user and the database, and finds that chorionic gonadotrophin contains dextran 40, so the information processing unit 2 derives a result from the analysis that extran 40 has allergenicity and those with an allergic constitution must undergo a skin test first to ensure safety, which deserves attention;
(4) Feedback of analysis results: the information processing unit 2 sends the analysis result to the window display unit 4 for the doctor's use as reference through the result feedback unit 3. The doctor analyzes the result and makes inquiries of the female drug user according to the early warning in the window display unit 4, suspends the prescription, arranges a skin test for the female drug user, and selects the Traditional Chinese Medicine of the prescription according to the result of the skin test.

### Embodiment 4

The embodiment relates to an analysis and management method for pharmaceutical excipients which is separately operated in the computer of pharmacies equipped with an analysis and management apparatus for pharmaceutical excipients, whose process includes four steps: creation of a database, input of the information on the drug user, analysis of the information on the drug user and feedback of analysis results:

(1) Creation of a database: the development personnel of the analysis and management apparatus for pharmaceutical excipients input the data on the drugs and pharmaceutical excipients currently used in the market into an information input unit 1 by scanning QR codes, entry or online download, the information input unit 1 sends the data above to the information processing unit 2 for recording and storage, the data above are regularly maintained, updated, modified, supplemented and deleted, and the data above are associated to realize the creation of a database;

(2) Input of the information on the drug user: the pharmacist inputs the prescription of the doctor's advice online into the information input unit 1. The information input unit 1 supports input by scanning QR codes, links and entry and provides a drop-down menu. The prescription of the doctor's advice for the drug user: the antiallergic prepared from Vitamin C powder for injection and calcium gluconate injection;

(3) Analysis of the information on the drug user: after receiving the information on the drug user, the information processing unit 2 makes an analysis and management by associating and comparing the information on the drug user and the database, and finds that Vitamin C powders contain anhydrous sodium carbonate, so the information processing unit 2 derives the result from the analysis that the anhydrous sodium carbonate contained in Vitamin C powders is incompatible with the calcium gluconate injection;

(4) Feedback of analysis results: the information processing unit 2 sends the analysis result to the window display unit 4 for the doctor's use as reference through the result feedback unit 3. The pharmacist analyzes the result and communicates with the doctor issuing the prescription in time to withdraw the prescription according to the early warning in the window display unit 4.

### Embodiment 5

The embodiment relates to an analysis and management method for pharmaceutical excipients which is realized stand-alone in the cell phone equipped with an analysis and management apparatus for pharmaceutical excipients, whose process includes four steps: creation of a database, input of the information on the drug user, analysis of the information on the drug user and feedback of analysis results:

(1) Creation of a database: the development personnel of the analysis and management apparatus for pharmaceutical excipients input the data on the drugs and pharmaceutical excipients currently used in the market into an information input unit 1 by scanning QR codes, entry or online download, the information input unit 1 sends the data above to the information processing unit 2 for recording and storage, the data above are regularly maintained, updated, modified, supplemented and deleted, and the data above are associated to realize the creation of a database;
(2) Input of the information on the drug user: the drug user inputs the information on the drugs bought by himself/herself into the information input unit 1 by scanning QR codes;
(3) Analysis of the information on the drug user: after receiving the information, the information processing unit 2 makes an analysis of the compatibility of pharmaceutical excipients and management of administration information, and finds the incompatibilities, administration information and dietetic contraindications of the pharmaceutical excipients;
(4) Feedback of analysis results: the information processing unit 2 sends the early warning to the window display unit 4 through the result feedback unit 3. The drug user administers drugs or goes to a medical organization and consults a doctor in time according to the analysis result in the window display unit 4.

Although the invention has been illustrated and described in greater detail with reference to the preferred exemplary embodiment, the invention is not limited to the examples disclosed, and further variations can be inferred by a person skilled in the art, without departing from the scope of protection of the invention.

For the sake of clarity, it is to be understood that the use of “a” or “an” throughout this application does not exclude a plurality, and “comprising” does not exclude other steps or elements.

1. An analysis and management method for pharmaceutical excipients wherein it is realized on-line or stand-alone in the server, computer, communication equipment or handheld mobile data terminals of the medical organization equipped with an analysis and management apparatus for pharmaceutical excipients, whose process includes four steps: creation of a database, input of the information on the drug user, analysis of the information on the drug user and feedback of analysis results:
(a) creation of a database: the data on drugs and pharmaceutical excipients are input into an information input unit by scanning QR codes, entry or online download, the information input unit sends the data above to the information processing unit for recording and storage, the data above is regularly maintained, updated, modified, supplemented and deleted, and the data above is associated to realize the creation of a database;
the data input include review, pharmaceutical research materials, dietetic contraindications of pharmaceutical excipients, and incompatibilities between pharmaceutical excipients as well as between pharmaceutical excipients and sovereign drugs; the review includes the name and nomenclature basis of drugs or pharmaceutical excipients, supporting documents, purposes and basis of subject determination, summary and evaluation of main research results, a sample, preparation instructions and latest references of inserts, and design samples of packages and labels; the pharmaceutical research materials include the review of the pharmaceutical research materials, the research materials and literature materials on production processes, the test materials and literature materials on confirmation of chemical structures or constituents, the test materials and literature materials on quality research, the compatibility test materials and literature materials on drugs, standard drafts and preparation instructions, standards or controls, the self-inspection reports of three consecutive lots of drugs, the test materials and literature materials on stability studies, the selection basis and quality standard constitution of packaging materials and containers in direct contact with pharmaceutical excipients, as well as periodical literature, newspaper reporting, research reports and Internet information covering the adverse consequences caused by the properties of pharmaceutical excipients;
wherein the name of drugs or pharmaceutical excipients includes a formal product name, chemical name, English name and Pinyin; the supporting documents include documents supporting the legal registration of the applicant, the copy of the Drug Production Licence, the description of the patent situation and ownership state of the prescription and processes of pharmaceutical excipients or used as well as a guarantee against the infringement of others patents; the purposes and basis of subject determination include foreign and domestic literature materials and situation review relevant to research and development, entry into the market and sales, production and application in preparations of this product; the summary and evaluation of the main research results include the summary of the main research results made by the applicant and the comprehensive evaluation of the reported variety in safety, efficacy and quality controllability; a sample, preparation instructions and the latest references of inserts include names, chemical structural formulae or molecular formulae, uses, precautions, packages, specifications, contents and the expiry date of pharmaceutical excipients and clear indication of the wording of “Pharmaceutical Excipients”; the review of the pharmaceutical research materials includes the overview of tests materials and foreign and domestic literature materials on synthesis processes, proscription screening, structure confirmation, quality research, quality standard development, stability study, etc.; the research materials and literature materials on production processes include the process flow, chemical reaction formulae, operation steps, starting materials, organic solvents, reaction conditions (temperature, pressure, time and catalysts) and operation steps of preparation, refinement methods, main physical and chemical constants as well as data accumulation results at different stages, indication of feeding amount and yield rate, possible impurities or other intermediate products produced or introduced during the processes, and provision of the specifications and standards of chemical raw materials used, the source, scientific name, preparation process and modification basis (where any main references are different) of animal-based, plant-based and mineral-based raw materials; the test materials and literature materials on quality research include physical and chemical constants, purity testing, content determination and methodological validation as well as data accumulation results at different stages; the test materials and literature materials on drug compatibility, standard drafts and preparation instructions, and standards or controls;
(b) input of the information on the drug user: the information on the drug user and the prescription or the drug name of the doctor's advice are input on-line or stand-alone into the information input unit which supports QR code scanning, links and entry and has a drop-down menu;
wherein the information on the drug user includes the physical examination report, past medical history, history of present illness, examination and treatment situations, description and parameters about conditions, and information about sovereign drugs and minister drugs of the used drugs of the drug user;
(c) analysis of the information on the drug user: after receiving the information on the drug user, the information processing unit searches the information on the sovereign drugs and minister drugs in the prescription or the drug of the doctor's advice, makes a comparison with the information in the database, analyzes whether there exist potential adverse consequences, and if there are any adverse consequence, searches in the database drug substitutes equivalent to the sovereign drugs in the prescription or the drug of the doctor's advice without changing the drug use intention, and makes an analysis to obtain an optimized safe plan of the prescription or the drug of the doctor's advice in which the sovereign drugs of the drug substitutes attain the drug-use intention and there doesn't exist any adverse consequence between the minister drugs of the drug substitutes and other prescriptions or drugs of the doctor's advice;
(d) feedback of analysis results: the information processing unit sends the optimized safe plan of the prescription or the drug of the doctor's advice obtained by analysis to the window display unit for the operator's use as reference through a result feedback unit.

2. The analysis and management apparatus for pharmaceutical excipients as claimed in claim 1, wherein the main structure of the analysis and management apparatus for pharmaceutical excipients comprises an information input unit, an information processing unit, a result feedback unit and a window display unit, wherein wireless electrical connection is adopted between the information input unit and the information processing unit, the information processing unit and the result feedback unit as well as the result feedback unit and the window display unit, the information input unit supports manual input, on-line input and input by scanning QR codes, the information processing unit records, stores, analyzes and manages the information input by the information input unit, the result feedback unit sends the information analyzed and processed by the information processing unit to the window display unit, and the window display unit shows the results fed back by the result feedback unit.
